(19) United States
(12) Patent Application Publication
(10) Pub. No.: US 2009/0238754 A1
(43) Pub. Date: Sep. 24, 2009

(54) USE OF PYRENE TO CARRY PEPTIDES ACROSS THE BLOOD BRAIN BARRIER

(75) Inventors: Renee WEGRZYN, Washington, DC (US); Andrew Nyborg, Gaithersburg, MD (US); D. Roxanne Duan, Bethesda, MD (US); Alan Rudolph, Potomac, MD (US)

Correspondence Address: FOLEY AND LARDNER LLP;SUITE 500, 3000 K STREET NW, WASHINGTON, DC, 20007, US

(73) Assignee: Adlyfe, Inc.

(21) Appl. No.: 12/363,254

(22) Filed: Jan. 30, 2009

Related U.S. Application Data
(60) Provisional application No. 61/038,634, filed on Mar. 21, 2008.

(51) Int. Cl. A61K 51/00; A61K 49/00; A61B 5/055; A61B 5/00; A61K 39/395; A61K 38/16; A61K 31/015

(52) U.S. Cl. 424/1.11; 424/9.1; 424/9.3; 424/9.6; 424/178.1; 514/12; 514/13; 514/765

(57) ABSTRACT

Described are methods for delivering a peptide agent across the blood-brain barrier, comprising administering to a subject a conjugate comprising (i) a peptide agent and pyrene, and related detection and therapeutic methods.

### RELATED APPLICATIONS

This application claims the benefit of priority to U.S. provisional application 61/038,634, filed Mar. 21, 2008, the entire contents of which are incorporated herein by reference in their entirety.

### FIELD OF THE INVENTION

The present invention relates generally to the field of delivering peptides, proteins and antibodies across the blood-brain barrier (BBB). More specifically, the present invention relates to methods for delivering peptides, proteins or antibodies across the BBB using pyrene-agent conjugates.

### BACKGROUND OF THE INVENTION

The detection and treatment of neurological conditions is often difficult due to the impermeability of endogenous and exogenously administered components to the brain as a result of the blood-brain barrier (BBB). The BBB effectively isolates the brain from peripheral agents such as peptides, proteins, large macromolecules, non-peptidic molecules, ions, and water-soluble non-electrolytes. For example, it is generally accepted that charged or hydrophilic molecules as well as molecules with a molecular weight greater than about 700 kDa do not cross the BBB. It is also generally accepted that peptides, such as peptides of about 21 amino acid residues, do not efficiently cross the BBB, nor do longer peptides such as the 40-residue Aβ40 protein and the 42-residue Aβ42 protein, both associated with Alzheimer's disease. Thus, the BBB prevents the delivery of detection agents as well as therapeutics, that otherwise, may be useful in the diagnosis and treatment of a variety of neurological disorders.

Prior attempts at effectively transporting agents to the brain have included conjugating agents to carrier moieties, using liposomal formulations, and using nanoparticles. Exemplary carrier moieties include naturally occurring polyamines (U.S. Pat. No. 5,670,477), carriers such as lysozyme, hemoglobin, cytochrome-c and substance-P (U.S. Pat. No. 5,604,198), and sugars (U.S. Pat. No. 5,260,308). Prior attempts at effectively transporting Aβ protein to the brain have used Aβ40 or smaller fragments, such as Aβ1-30, conjugated to a carrier such as OX26 or putrescine. The receptor for advanced glycation end products (RAGE) also has been proposed for mediating transport across the BBB, particularly for Aβ protein.

There remains a need, however, for methods, agents and kits for delivering peptide agents, including peptides, proteins and antibodies, across the BBB.

### SUMMARY OF THE INVENTION

In accordance with one embodiment, the invention provides a method for delivering a peptide conjugate across the blood brain barrier, comprising administering to a subject a conjugate comprising the peptide agent and pyrene. In some embodiments the peptide agent is a detection agent capable of identifying a protein or structure associated with a neurological disorder. In another embodiment, the peptide agent is a therapeutic agent useful in treating a neurological condition. In some embodiments, the peptide agent includes an amino acid sequence corresponding to a region of a target protein which undergoes a conformational shift from an alpha-helical conformation to a beta-sheet conformation, but does not include the full-length sequence of the target protein. In other embodiments the peptide agent is an antibody specific for a protein or structure associated with a neurological condition. In one embodiment, the conjugate further comprises a detectable label. In another embodiment the conjugate comprises a pyrene derivative, such as alkylated pyrene analogs, pyrene butyrate, PEGylated pyrene, pyrene-albumin analogs, pyrene derivatives comprising a free carboxyl group and pyrene derivatives comprising a free amine group. In some embodiments, the conjugate comprises two or more pyrene moieties.

In accordance with another embodiment, the invention provides an in vivo method of detection comprising administering to a subject a conjugate comprising a peptide detection agent and pyrene, and detecting conjugate that is localized in a subject's brain. In one embodiment, the detection agent is capable of identifying a protein or a structure associated with a neurological condition. In some embodiments, the conjugate comprises two or more pyrene moieties. In some embodiments, at least one pyrene moiety is a pyrene derivative comprising a free carboxyl group and at least one pyrene moiety is a pyrene derivative comprising a free amine group. In one embodiment, the pyrene is conjugated to the peptide detection agent at least at the N-terminus or C-terminus of the peptide, or at both the N- and C-termini of the peptide. In yet another embodiment, the detection agent is capable of identifying a protein in a specific conformation or state of self-aggregation. In another embodiment, the detection of localized conjugate involves detecting pyrene excimers.

In yet another embodiment, the invention provides an in vivo method of detection comprising administering to a subject a conjugate comprising peptide detection agent, pyrene and a detectable label, and detecting conjugate that has localized in the brain of the subject. In some embodiments the label is a fluorophore, MRI contrast agent, ion emitter, or a radioactive label.

In other embodiments, the invention provides a method for treating neurological conditions. The method comprises administering to a subject a therapeutically effective amount of a conjugate comprising a peptide therapeutic agent and pyrene. In one embodiment the peptide agent is an anti-amyloid agent.

### DESCRIPTION OF THE FIGURES

FIG. 1 shows the number of Aβ plaques detected per mm² by vehicle, peptide-agent pyrene conjugate, or pyrene butyrate administered intranasally to transgenic mice.

FIG. 2 illustrates the correlation between Aβ plaques detected in the cortex by intranasally administered conjugate (Aβ185) fluorescence (−) versus Thioflavin S staining (▴).

FIG. 3 illustrates the correlation between Aβ plaques detected in the cortex (FIG. 3A) and hippocampus (FIG. 3B) by intravenously administered conjugate (AD185) fluorescence (−) versus Thioflavin S staining (▪).

### DETAILED DESCRIPTION

Before particular embodiments of the invention are described and disclosed, it is to be understood that the particular materials, methods and compositions described herein are presented only by way of examples, and are not limiting of the scope of the invention. The technical and scientific terms used herein have the meanings commonly understood by one of ordinary skill in the art to which the present invention pertains, unless otherwise defined. Publications and other materials setting forth known methodologies to which reference is made are incorporated herein by reference in their entireties as though set forth in full.

Standard reference works setting forth the general principles of recombinant DNA technology include Sambrook, J., et al. (1989) Molecular Cloning: A Laboratory Manual, 2d Ed., Cold Spring Harbor Laboratory Press, Planview, N.Y.; McPherson, M. J. Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention. However, preferred materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any animal in need of detection or therapeutic treatment, including humans and domesticated animals, such as cats, dogs, swine, cattle, sheep, goats, horses, rabbits, and the like. “Subject” also includes animals used in research settings, including mice and other small mammals. A typical subject may be at risk of a neurological condition, disease or disorder or suspected of suffering from such a condition, or may be desirous of determining risk or status with respect to a particular condition. As used herein, “therapeutic” treatment includes the administration of a therapeutic agent to treat an existing condition, to prevent a condition that the subject is at risk or developing, or for health maintenance.

As used herein, the phrase “therapeutically effective amount” means that drug dosage in a subject that provides the specific pharmacological response for which the drug is administered in a patient in need of such treatment. It is emphasized that a therapeutically effective amount will not always be effective in treating the conditions/diseases described herein, even though such dosage is deemed to be a therapeutically effective amount by those of skill in the art.

As used herein, “peptide” refers to any polymer of two or more individual amino acids (whether or not naturally occurring) linked via a peptide bond. As used herein, the term “peptide agent” includes peptides, proteins, and antibodies. Peptides include fragments of full-length proteins, where fragments may include at least 5 contiguous amino acids, at least 10 contiguous amino acids, at least 15 contiguous amino acids, at least 20 contiguous amino acids, or at least 25 contiguous amino acids of the full-length protein. Peptides also include synthetic peptides.

As used herein, “conformation” or “conformational constraint” refers to the presence of a particular protein conformation, for example, an α-helix, parallel and antiparallel β-strands, a leucine zipper, a zinc finger, etc. In addition, conformational constraints may include amino acid sequence information without additional structural information. As an example, “-C-X-X-C-” is a conformational constraint indicating that two cysteine residues must be separated by two other amino acid residues, the identities of each of which are irrelevant in the context of this particular constraint. A “conformational change” is a change from one conformation to another.

The term “Aβ protein” is used herein to refer to all forms of the Aβ protein, including Aβ34, Aβ37, Aβ38, Aβ40 and Aβ42.

“Recombinant proteins or peptides” refer to proteins or peptides produced by recombinant DNA techniques, i.e., produced from cells, microbial or mammalian, transformed by an exogenous recombinant DNA expression construct encoding the desired protein or polypeptide. Proteins or peptides expressed in most bacterial cultures will typically be free of glycan. Proteins or peptides expressed in yeast may have a glycosylation pattern different from that expressed in mammalian cells.

As used herein, the term “naturally occurring” or “native” with reference to a peptide agent refer to agents (e.g., peptides, proteins and antibodies) that are present in the body or recovered from a source that occurs in nature. A native peptide agent may be modified either chemically or enzymatically, including post-translational modifications, including but not limited to, acetylation, glycosylation, phosphorylation, lipid conjugation, acylation and carbonylation.

As used herein, the term “synthetic” with reference to a peptide agent specifies that the agent is not naturally occurring, but may be obtained by other means such as chemical synthesis, biochemical methods, or recombinant methods.

The terms “analog,” “fragment,” “derivative,” and “variant,” when referring to peptides herein mean analogs, fragments, derivatives, and variants of such peptides that retain substantially similar functional activity or substantially the same biological function or activity as the reference peptides, as described herein. An “analog” includes a pro-polypeptide that comprises the amino acid sequence of a peptide.

A “fragment” is a portion of a peptide that retains substantially similar functional activity or substantially the same biological function or activity as the reference peptide, as shown in in vitro assays disclosed herein.

A “derivative” includes all modifications to a peptide of this invention that substantially preserve the functions disclosed herein and include additional structure and attendant function, e.g., PEGylated peptides or albumin fused peptides.

A “variant” includes peptides having an amino acid sequence sufficiently similar to the amino acid sequence of a reference peptide. The term “sufficiently similar’ means that the sequences have a common structural domain (e.g., sequence homology) and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Variants include peptides encoded by a polynucleotide that hybridizes to a complement of a polynucleotide encoding the reference polypeptide under stringent conditions. Such variants generally retain the functional activity of the reference peptides. Variants also include peptides that differ in amino acid sequence due to mutagenesis.

“Substantially similar functional activity” and “substantially the same biological function or activity” each means that the degree of biological activity is within about 50% to 100% or more, within 80% to 100% or more, or within about 90% to 100% or more, of that biological activity demonstrated by the reference peptide, when the biological activity of each peptide is determined by the same procedure or assay. For example, an analog or derivative of an may exhibit the same biological activity as the referent agent qualitatively, although it may exhibit greater or lesser activity quantitatively. The suitability of a given analog or derivative of an agent can be verified by routine screening methods to confirm that the analog or derivative exhibits an activity of interest that is substantially similar to that of the referent agent. An analog or derivative may possess additional structural features and/or exhibit additional functional properties, such as PEGylated agents, which comprise a PEG moiety and may exhibit a longer circulating half-life in vivo.

“Similarity” between two peptides is determined by comparing the amino acid sequences. An amino acid of one polypeptide is similar to the corresponding amino acid of a second polypeptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, P. (1989) EMBO J. 8:779-785. For example, amino acids belonging to one of the following groups represent conservative changes or substitutions:
Ala, Pro, Gly, Gln, Asn, Ser, Thr;
Cys, Ser, Tyr, Thr;
Val, Ile, Leu, Met, Ala, Phe;
Lys, Arg, His;
Phe, Tyr, Trp, His; and
Asp, Glu.

Some aspects of the invention relate to the diagnosis and treatment of diseases and conditions associated with a specific structural state of a protein, such as a specific conformation or self-aggregative state of a protein. PCT application PCT/US2007/016738 (WO 2008/013859) and U.S. patent application Ser. No. 11/828,953, which disclose relevant embodiments, are incorporated herein by reference in their entireties. Some aspects of the invention provide conjugates and methods for the in vivo detection of proteins in a specific structural state, including misfolded proteins and self-aggregated proteins, such as those associated with disease states, and conjugates and methods for the treatment of those disease states. In some embodiments, the proteins are associated with amyloidogenic diseases.

Proteins that are associated with human or animal disease when they adopt a specific conformational or self-aggregated state are known in the art. Examples of such diseases includes amyloidogenic diseases, including Alzheimer's disease (AD), cerebral amyloid angiopathy (CAA), and cerebral vascular disease (CVD). As used herein, “amyloidogenic diseases” are diseases in which amyloid plaques or amyloid deposits are formed in the body. Amyloid formation is found in a number of disorders, such as diabetes, AD, scrapie, bovine spongiform encephalopathy (BSE), Creutzfeldt-Jakob disease (CJD), chronic wasting disease (CWD), related transmissible spongiform encephalopathies (TSEs).

A variety of diseases are associated with a specific structural form of a protein (e.g., a “misfolded protein” or a self-aggregated protein), while the protein in a different structural form (e.g., a “normal protein”) is not harmful. In many cases, the normal protein is soluble, while the misfolded protein forms insoluble aggregates. Examples of such insoluble proteins include prions in transmissible spongiform encephalopathy (TSE); Aβ-peptide in amyloid plaques of Alzheimer's disease (AD), cerebral amyloid angiopathy (CAA), and cerebral vascular disease (CVD); α-synuclein deposits in Lewy bodies of Parkinson's disease, tau in neurofibrillary tangles in frontal temporal dementia and Pick's disease; superoxide dismutase in amylotrophic lateral sclerosis; and huntingtin in Huntington's disease. See, e.g., Glenner et al., J. Neurol. Sci. 94:1-28, 1989; Haan et al., Clin. Neurol. Neurosurg. 92(4):305-310, 1990.

Often, these insoluble proteins form aggregates composed of non-branching fibrils with the common characteristic of a β-pleated sheet conformation. In the CNS, amyloid can be present in cerebral and meningeal blood vessels (cerebrovascular deposits) and in brain parenchyma (plaques). Neuropathological studies in human and animal models indicate that cells proximal to amyloid deposits are disturbed in their normal functions. See, e.g., Mandybur, Acta Neuropathol. 78:329-331, 1989; Kawai et al., Brain Res. 623:142-146, 1993; Martin et al., Am. J. Pathol. 145:1348-1381, 1994; Kalaria et al., Neuroreport 6:477-80, 1995; Masliah et al., J. Neurosci. 16:5795-5811, 1996. Other studies additionally indicate that amyloid fibrils may actually initiate neurodegeneration. See, e.g., Lendon et al., J. Am. Med. Assoc. 277:825-831, 1997; Yankner, Nat. Med. 2:850-852, 1996; Selkoe, J. Biol. Chem. 271:18295-18298, 1996; Hardy, Trends Neurosci. 20:154-159, 1997.

While the underlying molecular mechanism that results in protein misfolding is not well understood, a common characteristic for all the above mentioned neurological disorders is the formation of fibrils which come together to form a β-sheet structure. Fibril formation and the subsequent formation of secondary β-sheet structures associated with plaque deposits, occurs via a complex mechanism involving a nucleation stage, in which monomers of the protein associate to form fibrils, followed by extension of the fibrils at each end. Thus, peptide, protein or antibody probes that are capable of disrupting fibril formation would prevent disease progression and thus be of therapeutic importance. Additionally, agents capable of associating with a particular self-associating state of the diseased protein are useful diagnostic tools to detect and quantify a particular form of the misfolded protein, as well as provide insights to the progression of the disease. Thus, highly selective peptide agents capable of associating with specific proteins in a particular state of self-aggregation are useful, both as detection agents as well as for therapeutic applications.

### A. Methods for Delivering Peptide Agents Across the BBB

Applicant has discovered that pharmaceutically relevant peptide agents, e.g., peptides, proteins and antibodies, conjugated to a pyrene carrier show an enhanced ability to cross the blood-brain barrier (BBB) when administered to a subject.

In one embodiment, there is provided a method for delivering a peptide agent across the BBB that comprises administering to a subject a conjugate comprising (i) a peptide agent and (ii) pyrene. In some embodiments, the peptide agent is a peptide, protein, or antibody. In some embodiments, the peptide agent is a detection agent or therapeutic agent. In specific embodiments, the peptide agent is a detection agent capable of identifying a target protein or structure (such as a specific conformation or state of self-aggregation) associated with a neurological condition. In other embodiments, the peptide agent is a therapeutic agent useful in treating a neurological condition. As used herein, “capable of identifying” means that the peptide agent selectively and preferentially binds to the target protein or structure.

The conjugate may be formulated in any composition suitable for administration to a subject, such as a composition comprising the conjugate and a pharmaceutically acceptable carrier. The conjugate may be administered by any suitable means, including by intranasal, intravenous, intraperitoneal, intraarterial, intramuscular, subcutaneous, oral, buccal, or transdermal, administration, and may be formulated accordingly. For example, the pharmaceutically acceptable carrier may be a liquid, so that the composition is adapted for parenteral administration, or may be solid, i.e., a capsule shell plus vehicle, a tablet, a pill and the like, formulated for oral administration. Alternatively, the pharmaceutically acceptable carrier may be in the form of a nebulizable liquid or solid so that the composition is adapted for inhalation. Pharmaceutically acceptable carriers are known in the art, and may include, without limitation, dissolution or suspension agents such as water or a naturally occurring vegetable oil like sesame, peanut, or cottonseed oil or a synthetic fatty vehicle like ethyl oleate or the like. Buffers, preservatives, antioxidants, binders, excipients, disintegrating agents, lubricants, sweetening agents and flavoring agents may also be included in the composition.

In the methods described herein, one or more conjugates comprising the same or different detection agents, therapeutic agents, pyrene moities and/or labels may be used, with each conjugate provided in the same composition or in one or more different compositions that may be administered simultaneously or sequentially by the same route or by one or more different routes.

In some embodiments, the pyrene-conjugated peptide agent exhibits a permeability across the BBB that is substantially greater than that of the non-conjugated active agent, such as at least three, at least five, at least ten, at least fifteen, at least twenty times greater, or more, than that of the non-conjugated active agent.

One measure of permeability across the BBB is the amount of conjugate that enters the brain relative to the amount that was injected and relative to the amount that enters other tissues (% IDI). In some embodiments, the pyrene-conjugate has an octanol/water partition coefficient between 1-10.

It is believed that some carriers that are used for increasing the permeability of a peptide across the BBB also have the effect of increasing the half-life of the peptide-carrier conjugate. For example, carriers that add a significant amount of structural size to the peptide-carrier conjugate may decrease the rate of degradation or clearance of the peptide. The Aβ40 peptide, for example, under normal physiological conditions is degraded in both the periphery and in the brain. However, conjugates using, for example, putrescine or OX26 as carriers increase the half life of Aβ40 dramatically. While an increased half-life may have some advantages, such as contributing to an increase in concentration in the brain, it also may have significant disadvantages, such as an increase in non-specific localization in the brain. This may be a particular concern if, for example, non-specifically localized conjugate contributes to a high background that decreases the sensitivity and/or selectivity of in vivo imaging.

The conjugates described herein do not suffer from this drawback. For example, experiments conducted with a conjugate comprising an Aβ peptide labeled at both termini with pyrene showed that the conjugate was cleared 6 hours post-administration, as determined by analysis of cerebrospinal fluid, which revealed no evidence of circulating conjugate.

The rate of localization and clearance or degradation of a conjugate can be assessed experimentally, for example, by administering the conjugates to mice and sacrificing them for analysis at different times post-administration, such as at time periods including 2 minutes, 10 minutes, 30 minutes, 60 minutes, 1 hour, 2 hours, 3 hours, 4 hours, 5 hours, 6 hours, or longer, post-administration.

The non-toxicity of the conjugates can be verified experimentally, for example, using in vitro assays and in vivo rodent toxicity studies that are known in the art.

### B. Peptide Agents

The nature of the peptide agent is not limited, other than comprising amino acid residues. The peptide agent can be a synthetic or a naturally occurring peptide, including a variant or derivative of a naturally occurring peptide. The peptide can be a linear peptide, cyclic peptide, constrained peptide, or a peptidomimetic. Methods for making cyclic peptides are well known in the art. For example, cyclization can be achieved in a head-to-tail manner, side chain to the N- or C-terminus residues, as well as cyclizations using linkers. The selectivity and activity of the cyclic peptide depends on the overall ring size of the cyclic peptide which controls its three dimensional structure. Cyclization thus provides a powerful tool for probing progression of disease states, as well as targeting specific self-aggregation states of diseased proteins.

In some embodiments, the peptide agent specifically binds to a target protein or structure associated with a neurological condition. In accordance with these embodiments, the invention provides agents useful for the selective targeting of a target protein or structure associated with a neurological condition, for diagnosis or therapy.

In some embodiments, the peptide agent is a peptide probe as described in PCT application PCT/US2007/016738 (WO 2008/013859) and U.S. patent application Ser. No. 11/828,953, the entire contents of which are incorporated herein by reference in their entirety. As described therein, such peptide probes may be useful as detection agents and/or as therapeutic agents. Exemplary peptide probes described in PCT application PCT/US2007/016738 (WO 2008/013859) and U.S. patent application Ser. No. 11/828,953 include an amino acid sequence corresponding to a region of the target protein which undergoes a conformational shift from an alpha-helical conformation to a beta-sheet conformation, and the peptide probe itself undergoes a conformational shift from an alpha-helical conformation to a beta-sheet conformation, but does not include the full-length sequence of the target protein. For example, a peptide probe may consist of at least 5, or from about 10 to about 25, contiguous amino acids from the target protein sequence, including at least 5, at least 10, up to about 25 and up to about 50, such as 5 to 50, 10 to 50, 5 to 25 or 10 to 25 contiguous amino acids from the target protein sequence. In some embodiments, the peptide probe may undergo a conformational shift when contacted with a target protein that is in the beta-sheet conformation.

As described in PCT application PCT/US2007/016738 (WO 2008/013859) and U.S. patent application Ser. No. 11/828,953, the peptide probes described therein are useful for detecting proteins in a sample or in vivo, and for detecting proteins in a specific structural state (e.g., a target structural state), such as a specific conformation or state of self-aggregation. For example, a peptide probe may be conjugated to pyrene such that it does not form excimers when the peptide probe is an alpha-helix or random coil conformation (or soluble state), but does form excimers when the peptide probe is in a beta-sheet conformation (or insoluble aggregated state). A target structural state may be associated with a disease while a different structural state is not associated with a disease. The target structural state may cause the disease, may be a factor in a symptom of the disease, may appear in a sample or in vivo as a result of other factors, or may otherwise be associated with the disease.

In some embodiments, the peptide agent comprises the amino acid sequence of SEQ ID NO 34 of PCT application PCT/US2007/016738 WO 2008/013859) and U.S. patent application Ser. No. 11/828,953. In some embodiments, the peptide agent comprises the amino acid sequence of SEQ ID NO:35, SEQ ID NO:36, SEQ ID NO:37, SEQ ID NO:38, or SEQ ID NO:45 of PCT application PCT/US2007/016738 WO 2008/013859) and U.S. patent application Ser. No. 11/828,953, which are useful in the context of the detection and treatment of AD. In some embodiments, the peptide agent is selected from SEQ ID NO:35, SEQ ID NO:36, SEQ ID NO:37, SEQ ID NO:38, or SEQ ID NO:45 of WO 2008/013859. In other embodiments, the peptide agent is other than SEQ ID NO 34, SEQ ID NO:35, SEQ ID NO:36, SEQ ID NO:37, SEQ ID NO:38, or SEQ ID NO:45 of WO 2008/013859. In some embodiments, the peptide is selected from SEQ ID NO:36 or SEQ ID NO:38 of WO 2008/013859. In some embodiments, the peptide is other than SEQ ID NO:36 or SEQ ID NO:38 of WO 2008/013859, including a peptide selected from SEQ ID NO 34, SEQ ID NO:35, SEQ ID NO:37, or SEQ ID NO:45 of WO 2008/013859 or another peptide. In some embodiments, the peptide is SEQ ID NO:36 of WO 2008/013859. In some embodiments, the peptide is other than SEQ ID NO:36 of WO 2008/013859, including a peptide selected from SEQ ID NO 34, SEQ ID NO:35, SEQ ID NO:37, or SEQ ID NO:45 of WO 2008/013859 or another peptide. In some embodiments, the peptide is SEQ ID NO:38 of WO 2008/013859. In some embodiments, the peptide is other than SEQ ID NO:38 of WO 2008/013859, including a peptide selected from SEQ ID NO 34, SEQ ID NO:35, SEQ ID NO:37, or SEQ ID NO:45 of WO 2008/013859 or another peptide.

(SEQ ID NO: 34 of WO 2008/013859)

SEQ ID NO: 1

Val Val Ala Gly Ala Ala Ala Ala Gly Ala Val His
Lys Leu Asn Thr Lys Pro Lys Leu Lys His Val Ala
Gly Ala Ala Ala Ala Gly Ala Val Lys
(SEQ ID NO: 35 of WO 2008/013859)

SEQ ID NO: 2

Leu Val Phe Phe Ala Glu Asp Val Gly Ser Asn Lys
Gly Ala Ile Ile Gly Leu Met
(SEQ ID NO:36 of WO 2008/013859)

SEQ ID NO: 3

Lys Leu Val Phe Phe Ala Glu Asp Val Gly Ser Asn
Lys Gly Ala Ile Ile Gly Leu Met
(SEQ ID NO: 37 of WO 2008/013859)

SEQ ID NO: 4

Leu Val Phe Phe Ala Glu Asp Val Gly Ser Asn Lys
Gly Ala Ile Ile Gly Leu Met Lys
(SEQ ID NO: 38 of WO 2008/013859)

SEQ ID NO: 5

Lys Leu Val Phe Phe Ala Glu Asp Val Gly Ser Asn
Lys Gly Ala Ile Ile Gly Leu Met Lys
(SEQ ID NO: 45 of WO 2008/013859)

SEQ ID NO: 6

Glu Val His His Gln Lys Leu Val Phe Phe Ala Glu
Asp Val Gly Ser Asn Lys Gly Ala Ile Ile Gly Leu
Met Val Gly Gly Val Val Ile Ala

In other embodiments, the peptide agent specifically binds to a target protein or structure associated with other neurological conditions, such as stroke, cerebrovascular disease, epilepsy, transmissible spongiform encephalopathy (TSE); Aβ-peptide in amyloid plaques of Alzheimer's disease (AD), cerebral amyloid angiopathy (CAA), and cerebral vascular disease (CVD); α-synuclein deposits in Lewy bodies of Parkinson's disease, tau in neurofibrillary tangles in frontal temporal dementia and Pick's disease; superoxide dismutase in amylotrophic lateral sclerosis; and Huntingtin in Huntington's disease and benign and cancerous brain tumors such as glioblastoma's, pituitary tumors, or meningiomas.

In some embodiments, the peptide agent undergoes a conformational shift other than the alpha-helical to beta-sheet shift discussed above, such as a beta-sheet to alpha-helical shift, an unstructured to beta-sheet shift, etc. Such peptide agents may undergo such conformational shifts upon interaction with target peptides or structures associated with a neurological condition.

In other embodiments, the peptide agent is an antibody that specifically binds to a target protein or structure associated with a neurological condition, such as a target protein or structure (such as a specific conformation or state of self-aggregation) associated with an amyloidogenic disease, such as the anti-amyloid antibody E610, and NG8. Other anti-amyloid antibodies are known in the art, as are antibodies that specifically bind to proteins or structures associated with other neurological conditions.

Other peptide detection agents include fluorescent proteins, such as Green Flourescent Protein (GFP), streptavidin, enzymes, enzyme substrates, and other peptide detection agents known in the art.

Exemplary peptide therapeutic agents include peptide macromolecules and small peptides. For example, neurotrophic proteins are useful as peptide agents in the context of the methods described herein. Neurotrophic proteins include nerve growth factor (NGF), brain-derived neurotrophic factor (BDNF), neurotrophin-3 (NT-3), neurotrophin-4 (NT-4), neurotrophin-5 (NT-5), insulin-like growth factors (IGF-I and IGF-II), glial cell line derived neurotrophic factor (GDNF), fibroblast growth factor (FGF), ciliary neurotrophic factor (CNTF), epidermal growth factor (EGF), glia-derived nexin (GDN), transforming growth factor (TGF-.alpha. and TGF-.beta.), interleukin, platelet-derived growth factor (PDGF) and S100β protein, as well as bioactive derivatives and analogues thereof.

Neuroactive peptides also include the subclasses of hypothalamic-releasing hormones, neurohypophyseal hormones, pituitary peptides, invertebrate peptides, gastrointestinal peptides, those peptides found in the heart—such as atrial naturetic peptide, and other neuroactive peptides.

The subclass of hypothalamic releasing hormones includes as suitable examples, thyrotropin-releasing hormones, gonadotropin-releasing hormone, somatostatins, corticotropin-releasing hormone and growth hormone-releasing hormone.

The subclass of neurohypophyseal hormones is exemplified by compounds such as vasopressin, oxytocin, and neurophysins.

The subclass of pituitary peptides is exemplified by adrenocorticotropic hormone, β-endorphin, α-melanocyte-stimulating hormone, prolactin, luteinizing hormone, growth hormone, and thyrotropin.

Suitable invertebrate peptides are exemplified by FMRF amide, hydra head activator, proctolin, small cardiac peptides, myomodulins, buccolins, egg-laying hormone and bag cell peptides.

Gastrointestinal peptides includes such neurologically active compounds such as vasoactive intestinal peptide, cholecystokinin, gastrin, neurotensin, methionineenkephalin, leucine-enkephalin, insulin and insulin-like growth factors I and II, glucagon, peptide histidine isoleucineamide, bombesin, motilin and secretins.

Examples of other neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

Peptide agents also include proteins associated with membranes of synaptic vesicles, such as calcium-binding proteins and other synaptic vesicle proteins. The subclass of calcium-binding proteins includes the cytoskeleton-associated proteins, such as caldesmon, annexins, calelectrin (mammalian), calelectrin (torpedo), calpactin I, calpactin complex, calpactin II, endonexin I, endonexin II, protein II, synexin I; and enzyme modulators, such as p65.

Other synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT1, rab 3A, and rab 3B.

Peptide agents also include α-, β- and γ-interferon, epoetin, Fligrastim, Sargramostin, CSF-GM, human-IL, TNF and other biotechnology drugs.

Peptide agents also include peptides, proteins and antibodies obtained using recombinant biotechnology methods.

Peptide agents also include “anti-amyloid agents” or “anti-amyloidogenic agents,” which directly or indirectly inhibit proteins from aggregating and/or forming amyloid plaques or deposits and/or promotes disaggregation or reduction of amyloid plaques or deposits. Anti-amyloid agents also include agents generally referred to in the art as “amyloid busters” or “plaque busters.” These include drugs which are peptidomimetic and interact with amyloid fibrils to slowly dissolve them. “Peptidomimetic” means that a biomolecule mimics the activity of another biologically active peptide molecule. “Amyloid busters” or “plaque busters” also include agents which absorb co-factors necessary for the amyloid fibrils to remain stable.

Anti-amyloid agents include antibodies and peptide probes, as described in PCT application PCT/US2007/016738 (WO 2008/013859) and U.S. patent application Ser. No. 11/828,953, the entire contents of which are incorporated herein by reference in their entirety. As described therein, a peptide probe for a given target protein specifically binds to that protein, and may preferentially bind to a specific structural form of the target protein. While not wanting to be bound by any theory, it is believed that binding of target protein by a peptide probe will prevent the formation of higher order assemblies of the target protein, thereby preventing or treating the disease associated with the target protein, and/or preventing further progression of the disease. For example, binding of a peptide probe to a monomer of the target protein will prevent self-aggregation of the target protein. Similarly, binding of a peptide probe to a soluble oligomer or an insoluble aggregate will prevent further aggregation and protofibril and fibril formation, while binding of a peptide probe to a protofibril or fibril will prevent further extension of that structure. In addition to blocking further aggregation, this binding also may shift the equilibrium back to a state more favorable to soluble monomers, further halting the progression of the disease and alleviating disease symptoms.

Those skilled in the art will recognize that many of the peptide agents described above as exemplary detection agents also are useful as therapeutic agents, and that many of the peptide agents described above as exemplary therapeutic agents also are useful as detection agents. Thus, these descriptors are in no way limiting.

In some embodiments, the peptide agent is a variant of a peptide agent described above, with one or more amino acid substitutions, additions, or deletions, such as one or more conservative amino acid substitutions, additions, or deletions, and/or one or more amino acid substitutions, additions, or deletions that further enhances the permeability of the conjugate across the BBB. For example amino acid substitutions, additions, or deletions that result in a more hydrophobic amino acid sequence may further enhance the permeability of the conjugate across the BBB.

### C. Pyrene

The pyrene can be pyrene or any pyrene derivative or analog that, when conjugated to a non-peptide agent improves the permeability of the agent across the BBB.

Pyrene consists of four fused benzene rings:

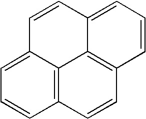

By “pyrene” deriviative or analog is meant a molecule comprising the four fused benzene rings of pyrene, wherein one or more of the pyrene carbon atoms is substituted or conjugated to a further moiety. Exemplary pyrene derivatives include alkylated pyrenes, wherein one or more of the pyrene carbon atoms is substituted with a linear or branched, substituted or unsubstituted, alkyl, alkenyl, alkynyl or acyl group, such as a C₁-C₂₀, linear or branched, substituted or unsubstituted alkyl, alkenyl, alkynyl or acyl group, where the group may be substituted with, for example, a moiety including an O, N or S atom (e.g., carbonyl, amine, sulfhydryl) or with a halogen. In some embodiments the pyrene derivative includes one or more free carboxyl groups and/or one or more free amine groups, each of which may be directly attached to a pyrene carbon atom or attached to any position on a linear or branched, substituted or unsubstituted, alkyl, alkenyl, alkynyl or acyl group as described above, such as being attached at a carbon atom that is separated from a pyrene carbon by 1 or more, such as 1 to 3, 1 to 5, or more, atoms. In some embodiments, the pyrene is substituted with one or more acetic acid moieties and/or one or more ethylamine moieties. In some embodiments, the pyrene derivative is substituted with a single methyl, ethyl, propyl or butyl group. In some embodiments, the pyrene is substituted with a short chain fatty acid, such as pyrene butyrate. In another embodiment, the pyrene is conjugated to albumin, transferring or an Fc fragment of an antibody. In some embodiments, the substituent is attached to pyrene through a carbon-carbon linkage, amino group, peptide bond, ether, thioether, disulfide, or an ester linkage.

Pyrene derivatives can be made by methods known in the art. For example, substituted pyrenes may be used to attach fatty acids to the tetracyclic scaffold. Suitable reagents, including functionalized alkyl derivatives of pyrene, and derivatizing reactions are known in the art. For example amino pyrene can be reacted with 1,4-butanedioic acid methyl ester to yield a butanoic acid derivative of pyrene. Alternatively, 1-thiocyanato pyrene can be reacted with 4-aminobuatnoic acid methyl ester to yield a thio-substituted butanoic acid derivative of pyrene. Yet other alternative reactions include reacting pyrene boronic acid and a substituted fatty acid to yield fatty acid derivatives of pyrene.

In other embodiments, the pyrene derivative is PEGylated pyrene, i.e, pyrene conjugated to polyethylene glycol (PEG). Such pyrene derivatives may exhibit a longer circulating half-life in vivo. In other embodiments, the pyrene derivative is pyrene conjugated to albumin.

In some embodiments, the pyrene derivative exhibits reduced toxicity as compared to pyrene. In some embodiments, the pyrene derivative exhibits an increased circulating half-life in vivo as compared to pyrene, such as PEGylated pyrene discussed above. In some embodiments, the pyrene derivate exhibits even greater increased permeability across the BBB as compared to pyrene, such as albumin conjugated pyrene. In some embodiments, the pyrene derivative has an octanol/water partition coefficient between 1-10.

### D. Conjugates

The peptide agent may be conjugated to pyrene by any means known in the art, including chemical (covalent) conjugation. In some embodiments, the peptide agent is directly conjugated to pyrene through a side chain residue. In one embodiment the pyrene is conjugated to the peptide agent via the ε-amino group of a lysine residue, Derivatives of pyrene, such as chloropyrene can be coupled to the ε-amino group of lysine through palladium catalyzed cross-coupling reactions. In other embodiments, the peptide agent is conjugated to pyrene through a linker. Compounds used as linkers are well known in the art, and include optionally substituted C₁-C₂₀ alkyl groups, alkanoic acids, alkenoic acids, alkynoic acids, alkoxide groups, aminoalkanoic acids, alkyl amines, alkoxy groups, bifunctional imido esters, glutaraldehyde, ethylene oxide polymers (PEG), optionally substituted aryl groups, alkynyl pyridyl, alkynyl bipyridyl, phthalic acid, malic acid and maleic acid, N-hydroxysuccinimide esters, hetero-bifunctional reagents and group specific-reactive agents such as the maleimido moiety, dithio moiety (SH) and carbodiimide moiety

Conjugates may be formed by chemical synthesis or bioengineering methods, such as methods including expressing pyrene in living organisms together with the agent. Such bioengineering methods include direct engineering of synthetic biological processes or evolution and screening for pyrene-agent conjugate combinations.

In some embodiments, the peptide agent is conjugated to a single pyrene moiety. In other embodiments, the peptide agent is conjugated to two or more pyrene moieties. When the peptide agent is conjugated to two or more pyrene moieties, each pyrene moiety may be conjugated to the agent (directly or through a linker).

In one embodiment the pyrene moiety is conjugated to the peptide agent at its N- or C-terminus. In another embodiment, the pyrene moiety is conjugated to the peptide agent at an internal (non-terminal) amino acid residue. In embodiments with two pyrene moieties, one pyrene moiety may be conjugated to each terminus of the peptide agent, one pyrene moiety may be conjugated to the N- or C-terminus and the other conjugated at an internal residue, or both may be conjugated at internal residues. When more than two pyrene moieties are conjugated to a peptide agent, the moieties can be positioned at any permutation or combination of terminal and internal residues. In some embodiments the pyrene moieties are conjugated in proximity to each other, while in others they are at spaced apart or distant positions on the peptide agent. In other embodiments, one or more pyrene moieties is conjugated (directly or through a linker) to one or more pyrene moieties, at least one of which is conjugated, directly or through a linker, to the peptide agent.

Regardless of the position(s) of the pyrene moiety(ies), the conjugate may exhibit enhanced permeability of the agent across the BBB.

In some embodiments, the conjugates are labeled with pyrene such that they are capable of forming pyrene excimers. That is, the peptide agents are conjugated to pyrene moieties in such a way as to permit excimer formation between pyrene moieties conjugated to the same or different molecules of peptide agent, as may be desired. In accordance with these embodiments, two or more pyrene moieties may be conjugated to the same peptide agent molecule so as to permit excimer formation by interaction between pyrene moieties on the same peptide agent molecule, such as may be associated, for example, with a specific conformation of the peptide agent. Alternatively, the excimer formation may be due to interaction between pyrene moieties on different peptide agent molecules, such as may be associated, for example, with localization, binding and/or interaction between the peptide agent molecules.

In some embodiments different pyrene derivatives are used, at least one of which includes one or more free carboxyl groups (such as an acetic acid moiety) and at least one of which includes one or more free amine groups (such as an ethylamine moiety), as discussed above. In accordance with this embodiment, interactions between the free carboxyl group(s) on one pyrene derivative and the free amine group(s) on another pyrene derivative may stabilize interactions between the pyrene derivatives, such as via the formation of a salt bridge, and may stabilize the excimer forming adducts and/or maximize excimer fluorescene. In accordance with these embodiments, two different pyrene derivatives may be conjugated to the same peptide agent molecule, such as to stabilize excimer formation by interaction between the different pyrene derivatives on the same peptide agent molecule, such as may be associated, for example, with a specific conformation of the peptide agent. Alternatively, one pyrene derivative may be conjugated to one peptide agent molecule and a different pyrene derivative may be conjugated to a different peptide agent molecule, such as to stabilize excimer formation by interaction between the different peptide agent molecules, such as may be associated, for example, with localization, binding and/or interaction between the peptide agent molecules.

In some embodiments, the conjugate is labeled with a detectable label. For example, the conjugate may comprise a peptide agent that is coupled or fused, either covalently or non-covalently, to a label. In embodiments where the peptide agent is a detection agent, the detectable label may offer improved detection or detection under additional conditions. In embodiments where the peptide agent is a therapeutic agent, the detectable label may offer detection in addition to the therapy offered by the therapeutic agent.

As used herein, a “detectable label” includes any moiety that can be detected. The specific label chosen may vary widely, depending upon the analytical technique to be used for analysis. The label may be complexed or covalently bonded at or near the amino or carboxy end of the peptide agent, which may be endcapped with a short, hydrophobic peptide sequence. In some aspects of the invention, both the amino and carboxy ends of the peptide agent are endcapped with small hydrophobic peptides ranging in size from about 1 to about 5 amino acids. These peptides may be natural or synthetic, but are often natural (i.e., derived from the target protein). A label may be attached at or near the amino and/or carboxy end of the peptide, or at any other suitable position.

As used herein, a “detectable label” is a chemical or biochemical moiety useful for labeling the conjugate. “Detectable labels” may include fluorescent agents (e.g., fluorophores, fluorescent proteins, fluorescent semiconductor nanocrystals), phosphorescent agents, chemiluminescent agents, chromogenic agents, quenching agents, dyes, radionuclides, metal ions, metal sols, ligands (e.g., biotin, streptavidin haptens, and the like), enzymes (e.g., beta-galactosidase, horseradish peroxidase, glucose oxidase, alkaline phosphatase, and the like), enzyme substrates, enzyme cofactors (e.g., NADPH), enzyme inhibitors, scintillation agents, inhibitors, magnetic particles, oligonucleotides, and other moieties known in the art.

Where the agent or label is a fluorophore, one or more characteristics of the fluorophore may be used to assess the state of the labeled conjugate. For example, the excitation wavelength of the fluorophore may differ based on whether the conjugate is bound or free. In some embodiments, the emission wavelength, intensity, or polarization of fluorescence also may vary based on the state of the conjugate.

As used herein, a “fluorophore” is a chemical group that may be excited by light to emit fluorescence or phosphorescence. A “quencher” is an agent that is capable of quenching a fluorescent signal from a fluorescent donor. A first fluorophore may emit a fluorescent signal that excites a second fluorophore. A first fluorophore may emit a signal that is quenched by a second fluorophore. The probes disclosed herein may undergo fluorescence resonance energy transfer (FRET).

Fluorophores and quenchers may include the following agent (or fluorophores and quenchers sold under the following tradenames): 1,5 IAEDANS; 1,8-ANS; umbelliferone (e.g., 4-Methylumbelliferone); acradimum esters, 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF₂ (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hep; Coelenterazine ip; Coelenterazine n; Coelenterazine 0; Coumarin Phalloidin; C-phycocyanine; CPM Methylcoumarin; CTC; CTC Formazan; Cy2™; Cy3.1 8; Cy3,5™; Cy3™; Cy5.1 8; Cy5.5™; Cy5™; Cy7 ™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630—NHS; DY-635—NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF₂); a fluorescent protein (e.g., GFP (S65T); GFP red shifted (rsGFP); GFP wild type, non-UV excitation (wtGFP); GFP wild type, UV excitation (wtGFP); and GFPuv); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; luminol, Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO—PRO-1; PO—PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Agents may include derivatives of fluorophores that have been modified to facilitate conjugation to another reactive molecule. As such, agents may include amine-reactive derivatives such as isothiocyanate derivatives and/or succinimidyl ester derivatives of the agent.

In embodiments for in vivo detection, agents useful for in vivo detection can be used. For example, agents useful for magnetic resonance imaging, such as fluorine-18 can be used, as can chemiluminescent agents.

In one embodiment, the label is a PET or an MRI image contrast agent. Although MRI was initially hoped to provide a means of making definitive diagnoses noninvasively, the addition of contrast agents in many cases improves the sensitivity and/or specificity towards the tissue being imaged. MRI contrast agents can include positive or negative agents. Positive agents generally include paramagnetic molecule or short-T1 relaxation agents, although the combination of the two are also used. Exemplars of paramagnetic, positive GI contrast agents include ferric chloride, ferric ammonium citrate, and gadolinium-DTPA (with and without mannitol). Short T1 relaxation time contrast agents include mineral oil, oil emulsions, and sucrose polyester. Diamagnetic agents are used as negative contrast agent, for example, a mixture of kaolin and bentonite. Another diamagnetic contrast agent is suspension of a barium sulfate. Additionally, perfluoro chemical agents, such as Perfluoroctylbromide (PFOB) can also be used as a negative MRI contrast agent. Superparamagnetic agents can be used as oral negative MRI contrast agents. Compounds such as magnetite albumin microspheres, oral magnetic particles (Nycomed A/S, Oslo, Norway), and superparamagnetic iron oxide (AMI121, Advanced Magnetics, Cambridge, Mass.) are generally used. These compounds contain small iron oxide crystals approximately 250 to 350 angstroms in diameter and are mixtures of Fe2O3 and Fe3O4.

In another embodiment, the agents is a radioactive agent. For example, the agent may provide positron emission of a sufficient energy to be detected by machines currently employed for this purpose. One example of such an entity comprises oxygen-15 (an isotope of oxygen that decays by positron emission). Another example are compounds having fluorine-18 such as F-18 fluoro-L-dopa (FDOPA), F-18 fluorotyrosine (FTYR), fluorodeoxyglucose (FDG) as well as compounds containing C₁₁ atoms, (e.g., C-11 methionine (MET).

As noted above, the probes may be comprised in fusion proteins that also include a fluorescent protein coupled at the N-terminus or C-terminus of the probe. The fluorescent protein may be coupled via a peptide linker as described in the art (U.S. Pat. No. 6,448,087; Wurth et al., J. Mol, Biol. 319:1279-1290 (2002); and Kim et al., J. Biol. Chem. 280:35059-35076 (2005), which are incorporated herein by reference in their entireties). In some embodiments, suitable linkers may be about 8-12 amino acids in length. In further embodiments, greater than about 75% of the amino acid residues of the linker are selected from serine, glycine, and alanine residues.

Detectable labels also include oligonucleotides. For example, the peptide probes may be coupled to an oligonucleotide tag which may be detected by known methods in the art (e.g., amplification assays such as PCR, TMA, b-DNA, NASBA, and the like).

Where the agent or label is a fluorophore, one or more characteristics of the fluorophore may be used to assess the state of the labeled conjugate. For example, the excitation wavelength of the fluorophore may differ based on whether the conjugate is bound or free. In some embodiments, the emission wavelength, intensity, or polarization of fluorescence also may vary based on the state of the conjugate.

E. In Vivo Detection with Peptide Conjugates

Also provided are in vivo detection (including in vivo imaging) methods for detecting conjugate that has crossed the BBB and localized in the brain. As used herein, “localized in the brain” means has crossed the blood brain barrier, and includes localization in fluid surrounding the brain.

In one embodiment, the method comprises (a) administering to a subject a conjugate comprising (i) a peptide detection agent and (ii) pyrene and (b) detecting conjugate that has localized in the brain of the subject. In some embodiments, the peptide detection agent specifically binds to a protein or structure localized in the brain, thereby providing selective targeting of the protein or structure. In some embodiments, the conjugate specifically binds to a protein or structure localized in the brain and associated with a neurological condition, such as misfolded Aβ protein or Aβ plaques associated with Alzheimer's Disease, or other proteins or structures associated with other neurological conditions, as discussed above, thereby providing selective targeting of the protein or structure.

In another embodiment, the method comprises (a) administering to a subject a conjugate comprising (i) a peptide agent and (ii) pyrene, wherein the conjugate is labeled with a detectable label, and (b) detecting conjugate that has localized in the brain of the subject. In some embodiments, the conjugate specifically binds to a protein or structure localized in the brain, such as a protein or structure associated with a neurological condition, such as misfolded Aβ protein or Aβ plaques associated with Alzheimer's Disease, or other proteins or structures associated with other neurological conditions, as discussed above, thereby providing selective targeting of the protein or structure.

For example, the detection agent or label may be a fluorophore, an MRI contrast agent, ion emitter (PET), radioactive (scintillation counter), and the like. The conjugate can be detected by means suitable for detecting the detection agent or label, such as Fourier transform infra-red, ultra-violet, MRI, PET, scintillation counter, or fluorescence, light scattering, fluorescence resonance energy transfer (FRET), fluorescence quenching, and various chromatographic methods routinely used by one of ordinary skill in the art.

In some embodiments, the detecting step includes detecting pyrene excimer formation. An excimer is an adduct that is not necessarily covalent and that is formed between a molecular entity that has been excited by a photon and an identical unexcited molecular entity. The adduct is transient in nature and exists until it fluoresces by emission of a photon. An excimer represents the interaction of two fluorophores that, upon excitation with light of a specific wavelength, emits light at a different wavelength, which is also different in magnitude from that emitted by either fluorophor acting alone. It is possible to recognize an excimer (or the formation of an excimer) by the production of a new fluorescent band at a wavelength that is longer than that of the usual emission spectrum. An excimer may be distinguished from fluorescence resonance energy transfer since the excitation spectrum is identical to that of the monomer. The formation of the excimer is dependent on the geometric alignment of the fluorophores and is heavily influenced by the distance between them.

In one embodiment, pyrene moieties are present at each terminus of the peptide agent and excimer formation between fluorophores is negligible as long as the overall peptide conformation is α-helix or random coil, but excimers are formed when the peptide agent undergoes a structural change (such as a conformational change) such that the pyrene moieties are brought into proximity with each other. Pyrene moieties present at other positions on the peptide also may be useful in this context, as long as excimer formation is conformation dependent. Further, the magnitude of excimer formation is directly related to the amount of protein analyte present. For example, when the peptide agent is a peptide probe as described in PCT application PCT/US2007/016738 (WO 2008/013859) and U.S. patent application Ser. No. 11/828,953, the peptide agent may undergo a conformation shift that leads to excimer formation when it comes into contact with or interacts with a target protein or structure, such as an amyloid protein in a β-sheet conformation or in a specific state of self-aggregation. Thus, the methods of the present invention permit detection and in vivo imaging of a target protein or structure in the brain by detecting excimer formation.

The formation of excimers may be detected by a change in optical properties. Such changes may be measured by known fluorimetric techniques, including UV, IR, CD, NMR, or fluorescence, among numerous others, depending upon the fluorophore attached to the probe. The magnitude of these changes in optical properties is directly related to the amount of conjugate that has adopted the structural state associated with the change, and is directly related to the amount of target protein or structure present.

The conjugates described herein also are useful in other in vivo detection methods. For example, the conjugates can be used to detect a target protein or structure (such as a specific conformation or state of self-aggregation) in any other in vivo site, such as any organ including the heart, lungs, liver, kidney, or any tissue. Specific areas of interest also may include vascular tissue or lymph tissue. The conjugates described herein also are useful in detecting and imaging a target protein or structure in intravial microscopy methods.

In some embodiments, conjugates comprising different fluorescent labels (such as, for example, GFP) can be used with the pyrene conjugates in FRET methodologies. Fluorescence resonance energy transfer (FRET) involves the radiationless transfer of energy from a “donor” fluorophore to an appropriately positioned “acceptor” fluorophore. The distance over which FRET can occur is limited to between 1-10 nm, and hence this technique is used to demonstrate whether two types of molecules, labeled with a donor-fluorophore and a receptor fluorophore, occur within 10 nm of each other. Measuring FRET by confocal imaging enables the intracellular locations of the molecular interaction to be determined.

FRET can occur when the emission spectrum of a donor fluorophore significantly overlaps (>30%) the absorption spectrum of an acceptor. The combination of CFP and YFP labelled fusion proteins has been widely used for FRET measurements in living cells. Other donor and acceptor fluorophore pairs which have been used for FRET include CFP and dsRED, BFP and GFP, GFP or YFP and dsRED, Cy3 and Cy5, Alexa488 and Alexa555, Alexa488 and Cy3, FITC and Rhodamine (TRITC), YFP and TRITC or Cy3.

In some embodiments, a conjugate comprises a peptide labeled with a pyrene moiety and another fluorophore, positioned such that FRET can occur when the peptide adopts a specific conformation, such as a β-sheet conformation, such as may occur when a peptide probe as described above interacts with a target protein or structure. Administration of such a conjugate to a subject permits the detection of localized conjugate by the detection of the FRET signal.

F. Therapy with Peptide Conjugates

Also provided are methods of treating neurological disorders that comprise delivering a therapeutic agent across the BBB. In one embodiment, the method comprises (a) administering to a subject a conjugate comprising (i) a peptide therapeutic agent and (ii) pyrene. In another embodiment, the conjugate is labeled with a detectable label, and the method further comprises detecting conjugate that has localized in the brain of the subject. In some embodiments, the peptide therapeutic agent is an anti-amyloid agent. In some embodiments, the method comprises administering a therapeutically effective amount of conjugate. In some embodiments, the conjugate specifically binds to a protein or structure localized in the brain, such as a protein or structure and associated with a neurological condition, such as misfolded Aβ protein or Aβ plaques associated with Alzheimer's Disease, or other proteins or structures associated with other neurological conditions, as discussed above, thereby providing selective targeting of the protein or structure.

### EXAMPLES

The following examples provide further illustration of the invention without being limiting.

### Example 1

The following illustrates the ability of peptide-pyrene conjugates to cross the BBB. Similar methodology can be used to confirm the suitability of a given conjugate for use in accordance with the methods described herein, and/or to confirm that the conjugate exhibits enhanced permeability across the BBB as compared to the non-conjugated agent.

The following illustrates the ability of peptide agent conjugates to target Aβ plaques (e.g., insoluble self-aggregates of Aβ protein associated with Alheimer's disease) in vivo. A peptide agent specific for Aβ corresponding to residues 16-35 of the Aβ protein (SEQ ID NO:3) with an added C-terminal lysine residue (e.g., SEQ ID NO:5) for conjugating pyrene, and labeled at each terminus with pyrene is used.

SEQ ID NO: 3:
Lys Leu Val Phe Phe Ala Glu Asp Val Gly Ser Asn
Lys Gly Ala Ile Ile Gly Leu Met
SEQ ID NO: 5:
Lys Leu Val Phe Phe Ala Glu Asp Val Gly Ser Asn
Lys Gly Ala Ile Ile Gly Leu Met Lys

In vivo studies use four homozygous hAPP751SL transgenic 10 month old mice and four littermate controls (siblings not carrying the transgene). The labeled peptide agent conjugate is administered intranasally, at 10 μl liquid per administration (at concentrations of from 0.1 to 2.0 mg/ml) with an administration interval of a planned half of an hour, adjusted according to the condition of the animal after treatment.

At the end of the treatment, mice are sacrificed and CSF and brains are extracted. (All mice are sedated by standard inhalation anaesthesia, Isofluran, Baxter).

Cerebrospinal fluid is obtained by blunt dissection and exposure of the foramen magnum. Upon exposure, a Pasteur pipette is inserted to the approximate depth of 0.3-1 mm into the foramen magnum. CSF is collected by suctioning and capillary action until flow fully ceases. CSF is immediately frozen and kept at −80° C. until use.

After CSF sampling, the stomach, stomach content and the brains are rapidly removed. Brains are hemisected, and the right hemisphere of all mice are immersion fixed in freshly produced 4% Paraformaldehyde/PBS (pH 7.4) for one hour at room temperature, and transferred to a 15% sucrose/PBS solution for 24 hours to ensure cryoprotection. Thereafter, brains are frozen in liquid isopentane on the next day and stored at −80° C. until used for histological investigations. The other brain half is immediately shock frozen in liquid isopentane for future use.

Images are recorded from transgenic mice treated with the highest dose of peptide agent conjugate and from control mice and from a transgenic vehicle control (e.g., the diluent used for the peptide agent conjugate) to confirm that the peptide agent conjugate crosses the blood-brain barrier (BBB), which it does.

To assess the specifity of staining by the peptide agent conjugate, fluorescence is excited using a UV-2A and B-1E filter of a microscope to detect probable auto-fluorescence in the lower spectrum. Fluorescent parts are recorded in the consecutive slice to ensure that impurity (e.g. dust) does not causes fluorescence. Transgenic slices are stained with ThioflavinS to assess plaque load.

As noted above, hAPP751_{SL} transgenic mice express hAPP in certain blood vessels in the periphery of the brain. The peptide agent conjugate binds to the amyloid and agglomerates outside the blood vessel in the brain. In the nontransgenic mice, the peptide agent conjugate reaches the olfactory bulb, but does not bind to a specifiable morphological structure.

### Example 2

The following example confirms the ability of the Aβ peptide-agent conjugate described above to selectively target Aβ plaques in the brain after intranasal administration.

Three groups of three hAPP transgenic mice were treated with vehicle (10% DMSO), the Aβ peptide-agent conjugate described above, or pyrene butyrate. Mice received three 10 μl injections at 20 minute intervals over a one hour period. Mice were sacrificed 6 hours later and tissues were collected. Flourescence in sagital sections was performed using fixed frozen tissue and a UV-2A fileter-equipped microscope. All plaque counts were performed on a digital images using Image-Pro-Plus software (Media Cybernetics, Inc., Bethesda, Md.).

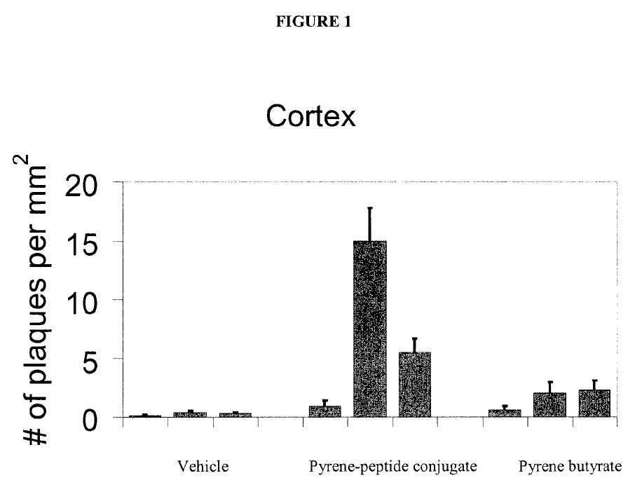

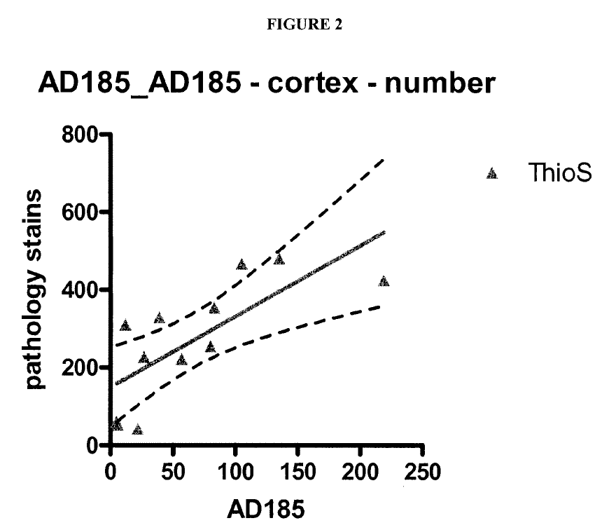

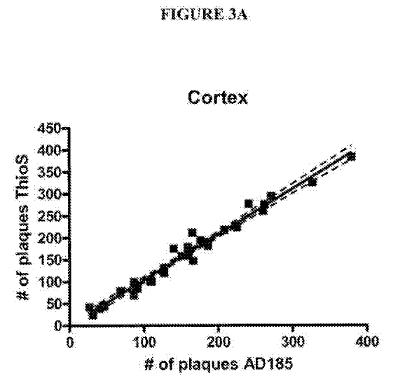

As seen in FIG. 1, only the mice treated with conjugate (“Pyrene-peptide conjugate”) showed fluorescent labeling of Aβ plaques, while mice treated with vehicle or pyrene butyrate did not. The mouse in the conjugate-treated group that displayed only background levels of fluorescence contained almost no Aβ plaques as determined by an anti-Aβ antibody (the 6E10 antibody), or Thioflavin S (which is specific for amyloid plaques) staining. FIG. 2 illustrates the correlation between conjugate fluorescence (AD185) and Thioflavin S staining. A positive correlation was found in both the hippocampus (data not shown) and cortex (plotted in FIG. 2), with an r2=0.555 and p=0.005.

Sequential sagital brain sections were stained with either 6E10 antibody or Thioflavin S and co-merged with fluorescent images from the conjugate-labeled sections. These data showed that the conjugate fluorescence coincided with the antibody and Thioflavin S plaque staining, further demonstrating the specificity of the conjugate for Aβ plaques.

### Example 3

The following example confirms the ability the Aβ peptide-agent conjugate described above to selectively target Aβ plaques in the brain after intravenous administration.

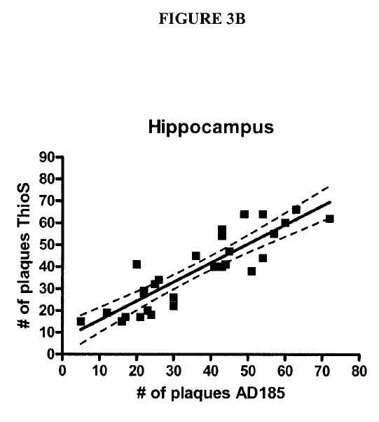

hAPP transgenic mice were administered the Aβ peptide-agent conjugate described above intravenously at a dose of 30 mg/kg through the tail vein. Mice were sacrificed at 6 hours after the administration of the conjugate, and brain sections were prepared for imaging as described above. After a section was imaged for conjugate fluorescence, it was bleached of fluorescence and stained with a Thioflavin S stain. The data revealed a significant correlation between conjugate fluorescence (AD185) and Thioflavin S staining, in both the cortex (FIG. 3A) and hippocampus (FIG. 3B).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with the true scope and spirit of the invention being indicated by the following claims.

What is claimed is:

1. A method for delivering a peptide agent across the blood-brain barrier, comprising administering to a subject a conjugate comprising:
peptide agent; and
pyrene.

2. The method of claim 1, wherein the peptide agent is a therapeutic agent or a detection agent.

3. The method of claim 2, wherein the peptide agent is capable of identifying a target protein associated with a neurological condition.

4. The method of claim 3, wherein the peptide agent selectively binds to a protein or structure associated with a neurological condition.

5. The method of claim 4, wherein the peptide agent includes an amino acid sequence corresponding to a region of the target protein which undergoes a conformational shift from an alpha-helical conformation to a beta-sheet conformation, but does not include the full-length sequence of the target protein.

6. The method of claim 5, wherein the detection agent comprises SEQ ID NO:2, SEQ ID NO:3, SEQ ID NO:4, SEQ ID NO:5 or SEQ ID NO:6.

7. The method of claim 4, wherein the peptide agent is an antibody specific for a protein or structure associated with a neurological condition.

8. The method of claim 2, wherein the peptide agent is a therapeutic agent useful for treating a neurological condition.

9. The method of claim 1, wherein the conjugate further comprises a detectable label.

10. The method of claim 1, wherein the pyrene is a derivative of pyrene.

11. The method of claim 9, wherein the derivative of pyrene is selected from the group consisting of alkyl pyrene, amino pyrene, pyrene carboxylate, pyrene butyrate, albumin-pyrene, PEGylated pyrene, a pyrene derivative comprising a free carboxyl group and a pyrene derivative comprising a free amine group.

12. The method of claim 1, wherein the conjugate comprises two or more pyrene moieties.

13. The method of claim 1, wherein conjugate exhibits enhanced permeability across the blood brain barrier as compared to the peptide.

14. An in vivo detection method comprising
(a) administering to a subject a conjugate comprising (i) a peptide detection agent and (ii) pyrene and
(b) detecting conjugate localized in the brain of the subject.

15. The method of claim 14, wherein the conjugate comprises two or more pyrene moieties.

16. The method of claim 14, wherein the peptide detection agent is conjugated to pyrene at a position selected from at least one of the C-terminus and the N-terminus of the peptide detection agent.

17. The method of claim 16, wherein the peptide detection agent is conjugated to pyrene moieties at each of the C-terminus and N-terminus of the peptide detection agent.

18. The method of claim 17, wherein step (b) comprises detecting pyrene excimer formation.

19. The method of claim 15, wherein at least one pyrene moiety is a pyrene derivative comprising a free carboxyl group and at least one pyrene moiety is a pyrene derivative comprising a free amine group.

20. The method of claim 14, wherein the peptide detection agent is capable of identifying a protein or structure associated with a neurological condition.

21. The method of claim 14, wherein peptide detection agent is capable of identifying a protein in a specific conformation or state of self-aggregation.

22. The method of claim 14, wherein the detection agent comprises SEQ ID NO:2, SEQ ID NO:3, SEQ ID NO:4, SEQ ID NO:5 or SEQ ID NO:6.

23. The method of claim 14, wherein the conjugate further comprises a detectable label.

24. The method of claim 23, wherein the label is selected from the group consisting of fluorophores, MRI contrast agents, ion emitters, and radioactive labels.

25. A method of treating a neurological condition, comprising administering to a subject in need thereof a therapeutically effective amount of a conjugate comprising (i) a peptide therapeutic agent and (ii) pyrene.

26. The method of claim 25, wherein the peptide therapeutic agent is useful in treating a neurological condition.

27. The method of claim 25, wherein the peptide therapeutic agent is an anti-amyloid agent.


SEQUENCE LISTING (6 sequences)

SEQ ID NO: 1 - protein, 33 aa, Artificial Sequence
  Description of Artificial Sequence Synthetic polypeptide
VVAGAAAAGAVHKLNTKPKLKHVAGAAAAGAVK

SEQ ID NO: 2 - protein, 19 aa, Artificial Sequence
  Description of Artificial Sequence Synthetic peptide
LVFFAEDVGSNKGAIIGLM

SEQ ID NO: 3 - protein, 20 aa, Artificial Sequence
  Description of Artificial Sequence Synthetic peptide
KLVFFAEDVGSNKGAIIGLM

SEQ ID NO: 4 - protein, 20 aa, Artificial Sequence
  Description of Artificial Sequence Synthetic peptide
LVFFAEDVGSNKGAIIGLMK

SEQ ID NO: 5 - protein, 21 aa, Artificial Sequence
  Description of Artificial Sequence Synthetic peptide
KLVFFAEDVGSNKGAIIGLMK

SEQ ID NO: 6 - protein, 32 aa, Artificial Sequence
  Description of Artificial Sequence Synthetic polypeptide
EVHHQKLVFFAEDVGSNKGAIIGLMVGGVVIA